(19) United States
(12) Patent Application Publication
(10) Pub. No.: US 2021/0310637 A1
(43) Pub. Date: Oct. 7, 2021

(54) ULTRA VIOLET DEVICE

(71) Applicant: Thomas C. YEE, Irvine, CA (US)

(72) Inventor: Thomas C. YEE, Irvine, CA (US)

(21) Appl. No.: 17/142,474

(22) Filed: Jan. 6, 2021

Related U.S. Application Data
(60) Provisional application No. 62/969,074, filed on Feb. 1, 2020.
(60) Provisional application No. 62/990,825, filed on Mar. 17, 2020.
(60) Provisional application No. 63/010,043, filed on Apr. 14, 2020.

(51) Int. Cl. F21V 14/02; A61L 2/10; A61L 2/24; F21V 21/26; F21V 21/15; F21V 7/00

(52) U.S. Cl. CPC F21V 14/025; A61L 2/10; A61L 2/24; F21Y 2115/10; F21V 21/15; F21V 7/0075; F21V 21/26

(57) ABSTRACT

A device includes lamps and LED devices emitting ultraviolet light mounted to movable arms. In an optional embodiment, a housing for the lamps includes shutters that close to redirect light from the lamps into an air passage and open to permit the movable arms to redirect the UV light into the environment. Optionally, the shutters, arms, and lamps are controlled by a controller.

### CROSS REFERENCE TO RELATED APPLICATIONS

This application claims priority to U.S. 62/990,825 entitled “ULTRAVIOLET DEVICE” filed Mar. 17, 2020; U.S. 62/969,074 entitled METHODS AND SYSTEMS FOR THE TREATMENT OF BLOOD TO TREAT DISEASES INCLUDING CORONAVIRUS″ filed Feb. 1, 2020; and U.S. 63/010,043 entitled “METHODS AND SYSTEMS FOR DISINFECTION USING ULTRAVIOLET LIGHT” filed Apr. 4, 2020. The content of these applications are incorporated by reference herein in their entirety.

### FIELD OF THE INVENTION

The present invention relates to sterilization and disinfection devices. More specifically, the present invention is a device that uses moving lamps emitting ultraviolet light to disinfect or sterilize surfaces in an area and a fan to deliver air past stationary lamps to disinfect or sterilize air in an area.

### BACKGROUND OF THE INVENTION

It is well known that microorganisms are responsible for infection and many diseases. The process of reducing the number of microorganisms in a sample (or area) is referred to as “disinfecting” while the process of eliminating all such microorganisms in a sample (or area) is referred to as “sterilizing.” Because of the cost, time, and/or danger of the methods required to sterilize an area, such as extreme high or low temperatures, ionizing radiation, anti-microbial chemicals, or the like, most areas, even in hospitals, doctors' offices, and other medical facilities, are merely disinfected rather than sterilized. For example, it is common for medical facilities such as hospitals and clinics to wipe the surfaces and floor in an operating room with a disinfectant between patients rather than “sterilizing” the surfaces and floor.

The drawback to disinfection is that the disinfectants, by design, are not guaranteed to eliminate all microorganisms but to merely reduce the number of microorganisms. Not only do the surviving microorganisms pose an inherent risk by their existence in an area such as the operating room of a medical facility, but over generations of using such disinfectants, the microorganisms that survive disinfection will tend to spawn microorganisms that are also disposed to survive disinfection. In this manner, microorganisms evolve genetically to resist the forms of disinfection used, and new disinfectants or new disinfection techniques are required to inactivate new strains of microorganisms.

Sterilization, on the other hand, totally eliminates the microorganisms from an area or surface. As one example, high heat that results from pressurized and heated water vapor is used to “sterilize” instruments in an autoclave. It is also known that ultraviolet (UV) light is effective in reducing or eliminating the number of microorganisms in air and water and on surfaces, depending on the intensity of the UV light and the exposure time. Specifically, it has been found that bacteria, bacterial spores, fungal spores, and viruses can be rendered inactive by exposure to UV light. It is known that UV light having a wavelength of 185 nanometers (nm) and 265 nm is absorbed by deoxyribonucleic acid (DNA). When the UV light is absorbed, T-T bonds formed by adjacent thymine molecules dimerize. These DNA defects can render the microorganism unable to reproduce and, therefore, inactive.

Currently, the world is in the grip of a pandemic caused by the Coronavirus (virus). That is, probably tens of millions of people have been exposed to the virus, over ten million people have become infected, over 502,000 people have died, and while there appear to be protocols that can mitigate and/or reduce the spread of the virus, and vaccines are being developed to prevent coronavirus infections from occurring, no medical or governmental authority is stating that we are past the point of danger. Indeed, many such medical experts and government leaders are warning of second waves of infections, as well as a surge associated with the cold winter months that could occur during the year.

There are several aspects of the virus that makes it particularly troubling. First is its rate of infection, or how easy it is to become infected and affected by the virus. The rate of infection, or ROI, is roughly about 2.1. This means for every single person infected by the virus, absent preventative measures, 2.1 other people will become sick. Secondly, the length of time between introduction and the appearance of symptoms can be up to 14 days. Third, many people can acquire the virus, but even after the 14 days “gestation” period, can be asymptomatic, meaning these people are unaffected and show no symptoms or only mild symptoms (which can be easily confused with a common cold or mild flu). Fourth, the severity of the illness brought about the virus is particularly problematic. For those that do catch the virus, and are particularly vulnerable to it, the affects can be devastating. Aside from the normal flu-like symptoms that people experience, lungs fill with fluid and it becomes exceedingly difficult to breathe. Ventilators are needed to assist many people in breathing. The mortality rate of patients who need assisted breathing with ventilators is extremely high, and ranges from 50-90%. According to competent medical authorities, a patient that needs the use of a ventilator is “unbelievably” sick. And in addition, for many people, complete recovery does not occur, and/or can take a significant amount of time, and can leave them with complications such as impaired cognitive ability, speech impediments, muscular and lung problems, among other issues.

In view of such dire statistics and mortality rates, most governments of the world instituted “preventative measures” to slow down the rate of transmission of the virus. In effect, people all over the world were requested to “stay in place.” That is, stay home, close businesses, stop traveling, and go out as little as possible. The world economy, as a result, has literally coming to a screeching halt. Oil prices have dropped to unheard of prices—for at least one day, traders were paying others to take the oil before it was delivered to them. Massive unemployment has occurred not only in the United States, but in many other countries. The economic affects have been, in short, overwhelming, and unbelievable. Nothing like this has ever happened so quickly to any economy ever.

The “preventative measures” described above included the “stay in place” orders, business shutdowns, and many other such measures. However, most rationale people have begun to realize no country or people can “stay in place” forever. It is a simple fact of nature that the virus is not going away, and that the “cure could be worse than the disease.” In addition, as time passes and scientists, doctors, and governments learn more about how the virus behaves, it has become very apparent that this virus affects different people in vastly different ways. That is, certain people and/or groups of people exhibit “comorbidities” that make them particularly susceptible to the effects of the virus, while many others, indeed the grand majority of others, will experience little to no symptoms, let alone be in danger of dying.

Nonetheless, no one wants to experience what the majority of the world went through from March of 2020 through the middle of the Summer of 2020. To that end, it has become apparent that one of the best practices that people can perform, is to prevent the spread of the coronavirus and other infectious diseases—that is, an ounce of prevention is worth a pound of cure.

Accordingly, a need has arisen for systems, methods, and modes for a device that uses moving lamps emitting ultraviolet light to disinfect or sterilize surfaces in an area and a fan to deliver air past stationary lamps to disinfect or sterilize air in an area.

### SUMMARY OF THE INVENTION

A device includes a housing. Optionally, the housing is mounted to a carriage. In an optional embodiment, the housing includes shutters. The shutters are movable between open and closed positions. The shutters expose lamps emitting UV light when open and substantially shield the lamps when closed. In an optional embodiment, the shutters direct UV light into an air passage when closed. Optionally, the lamps are adapted to emit UV light having a wavelength of substantially 254 nm.

At least one of the lamps is mounted to a movable arm that redirects the UV light emitted from the lamp. In an optional embodiment, the lamp is moveable between an orientation in which the UV light is emitted substantially horizontally from the housing and an orientation in which the UV light is emitted substantially vertically downward. In one optional embodiment, each lamp mounted to a moveable arm is moved between the same orientations. In an alternate or additional embodiment, different lamps may be movable between different orientations. In an optional embodiment, some lamps are stationary even if others are mounted to movable arms.

The lamps communicate with a controller. In one optional embodiment, the controller controls activation and deactivation of the lamps, the positions of the shutters, and the movement of the arms (and consequently the orientation of the lamps mounted to the arms). In one such optional embodiment, the controller includes a timer and a switch. In one such optional embodiment, the controller is programmed to activate the lamps during a time period measured by the timer and deactivate the lamps after the time period measured by the timer expires. In a further optional embodiment, the controller is programmed to control the movement of the arms based on the time period measured by the timer to complete a programmed set of movements during the time period.

In one optional embodiment, the controller includes a remote activation system. In one such optional embodiment, the remote activation system includes a wireless receiver local to the device and in communication with the controller. The controller is programmed to actuate a switch in response to a wireless signal received at the wireless receiver. The remote activation system further includes a wireless transmitter that transmits a wireless signal. Optionally, the wireless signal includes an activation signal and a deactivation signal. In such an optional embodiment, receipt of an activation signal at the wireless receiver causes the controller to close a switch to activate the lamps and receipt of a deactivation signal causes the controller to open a switch to deactivate the lamps.

In an optional embodiment, the controller includes a shut off system. In one such optional embodiment, the shut off system includes a motion detector and a switch connected to the lamps. Optionally, the controller is programmed to open the switch, thereby deactivating the lamps, when the motion detector detects motion and maintain the switch closed when no motion is detected.

In an additional or alternate optional embodiment, the controller includes a warning system. In one such optional embodiment, the warning system includes lights or lamps that flash or otherwise warn of the activation or impending activation of the lamps. Alternatively, or additionally, the warning system includes speakers and a storage device. In one such optional embodiment, the controller retrieves warning sounds, optionally recorded warning messages, from the storage device and controls the speakers to produce the warning sounds.

Optionally, the housing includes an air passage which has an air inlet that communicates with ambient air, an air passage that is exposed to UV light produced by the lamps when the lamps are in a position in the housing, and an air outlet that communicates with ambient air. In one such optional embodiment, the device includes a fan to move air through the air passage. Optionally, the fan may be controlled by the controller. For example, the controller may be programmed to close the shutters, if they are not already closed, and actuate the fan and the lamps when a command is received. In one such optional embodiment, the command may be transmitted from the wireless transmitter and received through the wireless receiver. In this manner, ambient air may be drawn through the air inlet, exposed to UV light from the lamps as the air passes through the air passage, and ejected from the device by the fan.

Optionally the device is configured to be mounted on a Roomba or moveable base or platform that can move around a room, such as an operating room. Movement is important because movement of the device allows it to clean a room with UV light.

Optionally, the device has an internal air tunnel that draws in air from the bottom of the device. The device has lamps and/or LED lights plastered through the middle of the internal air tunnel. In this manner, the air can be circulated and cleaned with UV light without the device having to move about the room.

Optionally, the device provides UV cleaning of air throughout a room, such as an operating room.

Optionally, the device continuously cleans the air in a public place, operating room, house, or other such location that is desired by a user.

### BRIEF DESCRIPTION OF THE DRAWINGS

FIG. 1 is a schematic diagram of a Portable ultra-violet germicidal irradiation (UVGI) System (UVGI system) according to an embodiment of the present invention.

FIG. 2 is a schematic diagram of a controller for use in the UVGI system according to an embodiment of the present invention.

FIGS. 3A through 3E are a series of schematic diagrams of the UVGI system shown in FIG. 1 according to an embodiment of the present invention.

FIGS. 4A and 4B are a series of schematic diagrams of the UVGI system shown in FIG. 1 according to an embodiment of the present invention.

FIGS. 5A through 5C are a series of schematic diagrams of the UVGI system shown in FIG. 1 according to an embodiment of the present invention.

FIGS. 6A through 6C are a magnified front view of the upper end of the arms of UVGI system shown in FIG. 1 according to an embodiment of the present invention.

FIG. 7 illustrates a control panel for use with the UVGI system shown in FIG. 1 according to an embodiment of the present invention.

FIGS. 8-13 are schematic views of the UVGI system shown in FIG. 1 according to an embodiment of the present invention in a room such as an operating room.

FIG. 14 is an illustration of a the UVGI system shown in FIG. 1 according to an embodiment of the present invention in a “non-operating” or “off” condition while located in an operating room.

FIGS. 15-16 are illustrations of the UVGI system shown in FIG. 1 according to an embodiment of the present invention in an “operating” or “on” conditions while located in an operating room.

FIG. 17 illustrates an autonomous and radio controlled base capable of moving the UVGI system shown in FIG. 1 according to an embodiment of the present invention on wheels.

FIG. 18A illustrates a partial front view of UV directional detection and avoidance system (system) 1800 that substantially prevents UVGI system as shown in FIG. 1 from exposing humans to UV light, and FIG. 18B illustrates a top view of a UV directional detection and avoidance system according to aspects of the embodiments.

FIG. 19 shows the UVGI system shown in FIG. 1 according to an embodiment of the present invention with an internal air tunnel with UV light and high efficiency particulate air (HEPA) filter and ventilation fan to clean the room air.

FIG. 20 illustrates a close-up view of a static UV lamp for use in the UVGI system shown in FIG. 1 according to an embodiment of the present invention.

FIG. 21 is a partial cross sectional view of the UVGI system shown in FIG. 1 illustrating an alternate embodiment in which air is pulled into an interior air chamber of the UVGI system by one or more fans and UV light from internally directed lamps is used to disinfect/sterilize the air according to an embodiment of the present invention.

FIG. 22 illustrates a perspective view of a UVGI system that includes additional features according to aspects of the embodiments.

FIG. 23 illustrates a top view of the UVGI system as shown in FIG. 22 that includes further additional features according to aspects of the embodiments.

FIG. 24A illustrates a top partial view of the UVGI Systems of FIGS. 1 and 22 with a movable shutter system in a closed condition according to aspects of the embodiments.

FIG. 24B illustrates a top partial view of the UVGI Systems of FIGS. 1 and 22 with a movable shutter system in an open condition according to aspects of the embodiments.

FIG. 25 illustrates a partial exploded view along lines A-A as shown in FIG. 3A according to aspects of the embodiments.

FIG. 26 illustrates a partial exploded view along lines B-B as shown in FIG. 3B according to aspects of the embodiments.

### DETAILED DESCRIPTION OF THE INVENTION

This application incorporates by references the information set forth in Appendices 1-4 in their entirety.

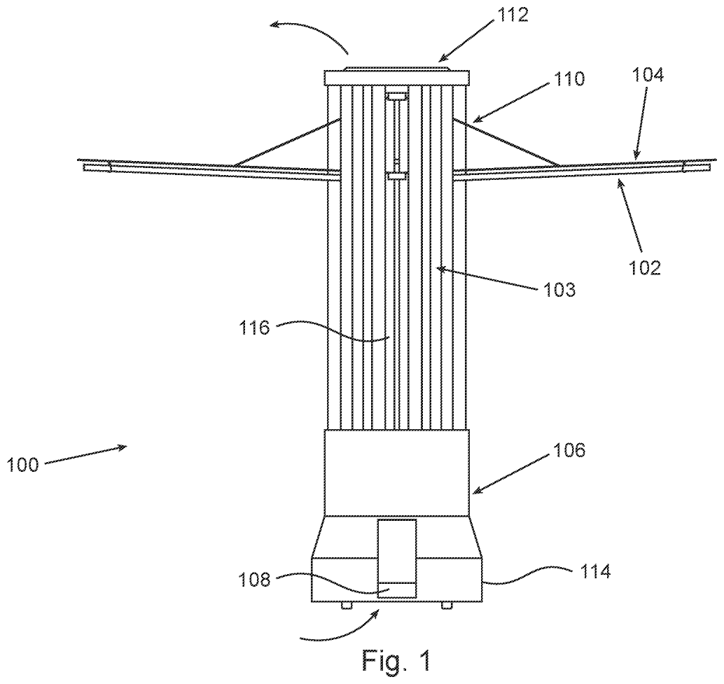

Reference is now made to the Figures wherein like parts are referred to by like numerals throughout. Referring generally to the Figures, and in particular to FIG. 1, in which a portable ultra-violet germicidal irradiation (UVGI) System (UVGI system) 100 according to an embodiment of the present invention is shown, a sterilization and disinfection device (UVGI system 100) may include two modes, with one mode directed toward surface microbes, e.g. bacteria, bacterial spores, fungi and fungal spores (including yeasts and molds and their spores), viruses, and the like, and another mode directed toward airborne microbes.

List of Elements in the Figures

| The following is a list of elements used in the Figures | |
|---|---|
| in numerical order: | |
| 100 | Ultra-Violet Germicidal Irradiation |
| | (UVGI) System (UVGI System) |
| 102 | Dynamic UV-C Bulb (Lamp) |
| 103 | Static UV-C Bulb |
| 104 | Extendable Bulb Retention Arms (Extendable Arms) |
| 106 | Main Body of UVGI System (Body/Housing) |
| 108 | Control Panel |
| 110 | Bracket Extension System |
| 112 | Fan |
| 114 | Base |
| 116 | Central Air Passage |
| 302 | Movable Shutter (Shutter) |
| 304 | Fixed Slat (Slat) |
| 702 | Lamp Status Indicators |
| 704 | Motion Sensor Display |
| 706 | Motion Sensor Switch |
| 708 | Power Switch |
| 710 | Timer Switch |
| 712 | Audio Volume Switch |
| 714 | Message Record Button |
| 716 | Motor Lift Speed |
| 718 | Microphone Jack |
| 800 | Room |
| 802 | Floor |
| 804 | Cabinet/Furniture |
| 806 | Wall(s) |
| 808 | Ceiling |
| 902 | Door |
| 1400 | Surgical Suite |
| 1402 | Surgical/Medical Equipment |
| 1702 | Programmable Remotely Controlled Self- |
| | Propelled Carriage (Carriage) |
| 1800 | Directional Detection and Avoidance System |
| 1804 | Infra-red (IR) Sensor |
| 1902 | High Efficiency Particulate Air (HEPA) Filter |
| 2002 | Parabolic UV Light Reflector (Reflector) |
| 2200 | UVGI System |
| 2202 | Internal Central Core Air Tunnel (Tunnel) |
| 2204 | Reflective Inner Surface of Air Tunnel |
| | (Reflective Inner Surface) |
| 2206 | Heat Switch |
| 2402 | Shutter Hinge |
| 2404 | Shutter Motor |

### LIST OF ACRONYMS USED IN THE SPECIFICATION

The following is a list of acronyms used in the specification in alphabetical order:

| | |
|---|---|
| DNA | Deoxyribonucleic Acid |
| eV | Electron Volts |
| HEPA | High Efficiency Particulate Air |
| IR | Infra-Red |
| LED | Light Emitting Diode |
| NFC | Near Field Communications |
| nm | Nanometers |
| UV | Ultra-Violet |
| UVGI | Ultra-Violet Germicidal Irradiation |

According to aspects of the embodiments, UVGI system 100 comprises one or more of the following: either or both dynamic (i.e., those that move) UV lamps (lamps) 102 and static (i.e., stationary) UV lamps (lamps) 103, extendable bulb retention arms (extendable arms) 104, main body/housing of UVGI system (housing) 106, control panel 108, bracket extension system 110, fan 112, base 114, central air passage 116, shutters 302, slats 304, programmable remotely controlled self-propelled carriage (carriage) 1702, and parabolic UV light reflectors (reflectors) 2002: each of these components will be described in greater detail below.

In certain embodiments, UVGI system 100 includes an efficient way to clean a room with UV light.

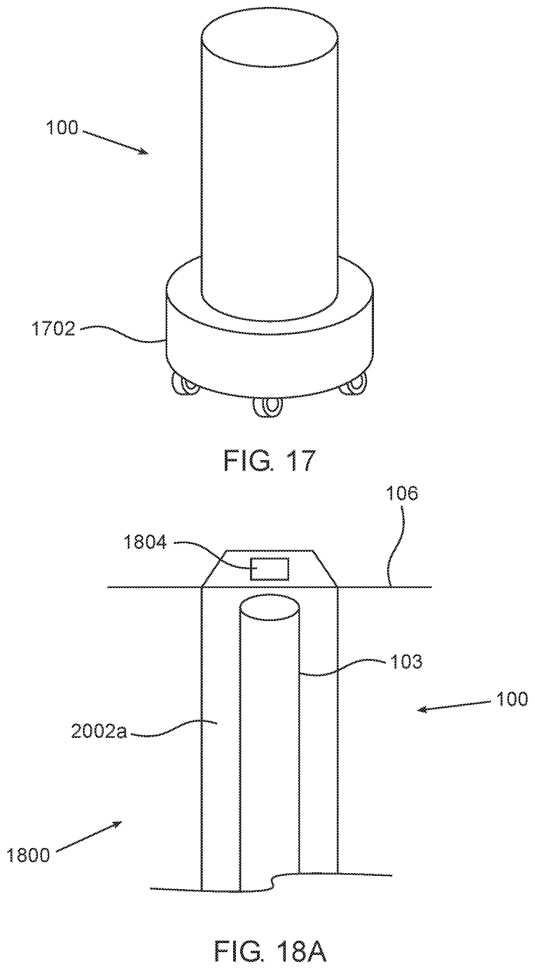

In certain embodiments, UVGI system 100 includes a base 114 and carriage 1702 (shown in FIG. 17) similar to Roomba automatic vacuum cleaner that can move in a room, with an infrared (IR) sensor that can detect presence of people to activate emergency shut off of the light.

In certain embodiments, UVGI system 100 includes a smart carriage 1702 that once the human operator pushes a button to record the path in a room, as the operator pushes the machine along that path for the first time, and names that path, the machine will remember that path.

In certain embodiments, UVGI system 100 will be able to follow the path and move along slowly.

In certain embodiments, an operator of UVGI system 100 can program how many minutes to shine UV light in a room.

In certain embodiments, UVGI system 100 will have extendable arms 104 that can hold dynamic UV LEDs/bulbs/lamps (lamps) 102. In certain embodiments, UVGI system 100 will not have extendable arms 104, but will have one or more internal light tunnels for continuous air cleaning and filtering while the light is shielded from outside. In certain embodiments, UVGI system 100 has both lamps 102 and 103 (as shown in FIG. 1, among other Figures)

In certain embodiments, UVGI system 100 has a heavy and high capacity battery at the base 114 so once charged it can operate for hours without being plugged in. This enables to mobility, and provides a heavy base 114 for better stability.

In certain embodiments, UVGI system 100 has a cylindrical shape.

In certain embodiments, UVGI system 100 is about 190 cm tall, has an outer cylinder diameter of about 30 cm, an internal air tunnel diameter of about 18 cm, and a base 114 diameter of about 50 cm.

In certain embodiments, UVGI system 100 is constructed with a substantially reflective aluminum surface to better reflect UV light and to prevent bacterial growth on the surface.

In certain embodiments, the entire surface of UVGI system 100, even around the base 114, is covered by UV LEDs, in the form of light strips, among other forms, such that substantially the entire exterior surface and the surface of the internal air tunnel of UVGI system 100 is covered by UV light producing devices.

In certain embodiments, UVGI system 100 is controlled via a remote wireless control panel.

In certain embodiments, UVGI system 100 will stop and change direction if the base 114 touches anything, or if the movement is impeded somehow such as upper structure touches anything.

Referring to FIG. 1, UVGI system 100 includes a housing 106. As discussed in greater detail below, the housing 106 may function to maintain lamps 102 (lamps 102, 103 can include UV bulbs and/or UV light emitting diodes (LEDs)) in a safe configuration when UVGI system 100 is not in use or in use to disinfect/sterilize air. Thus, in one such optional embodiment, the housing 106 may be configured with tolerances to substantially prevent leakage of ultraviolet (“UV”) light.

The housing 106 may be stationary or portable. In one optional embodiment, the housing 106 may be ported or carried by hand. In the optional embodiment illustrated in the Figures, the housing 106 is mounted to a carriage 1702 or base 114. In the optional embodiment illustrated in the Figures, the base 114 includes wheels and a push rail. As may be appreciated, in alternate optional embodiments, other mechanisms may be provided to transport UVGI system 100, such as tracks or the like. It is contemplated that the base 14 may be manual or may be motorized. That is, in alternate optional embodiments, the base 114 may include an electric motor, engine, or other means for delivering power to the wheels, tracks, or the like, as shown in FIG. 17, and denoted carriage 1702. Optionally, the housing 106 may include a power supply for the carriage 1702 or the carriage 1702 may receive power from an external source, such as electrical socket. In an optional embodiment, UVGI system 100 is bottom-heavy to reduce the risk of tipping.

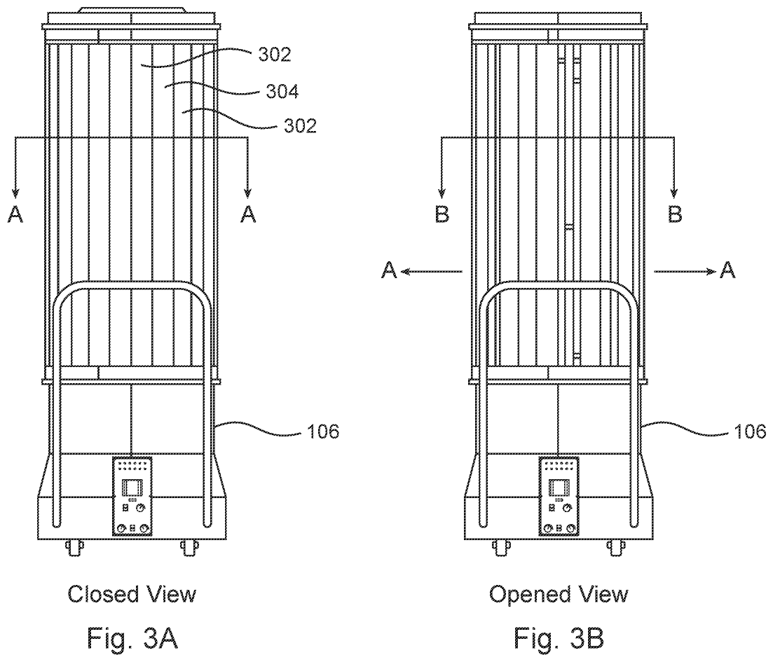

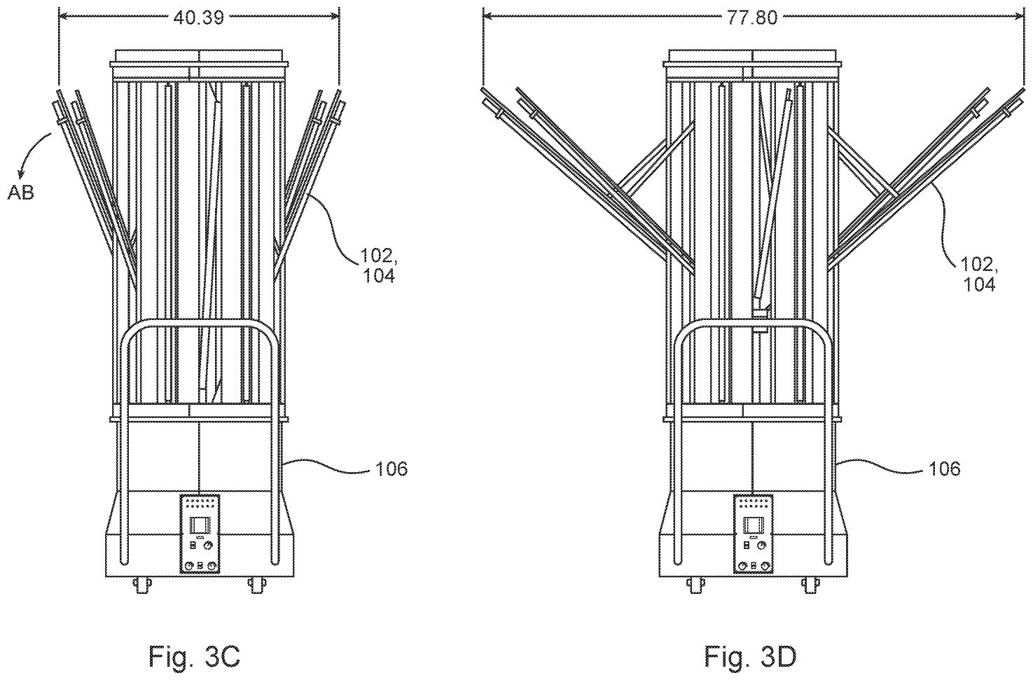

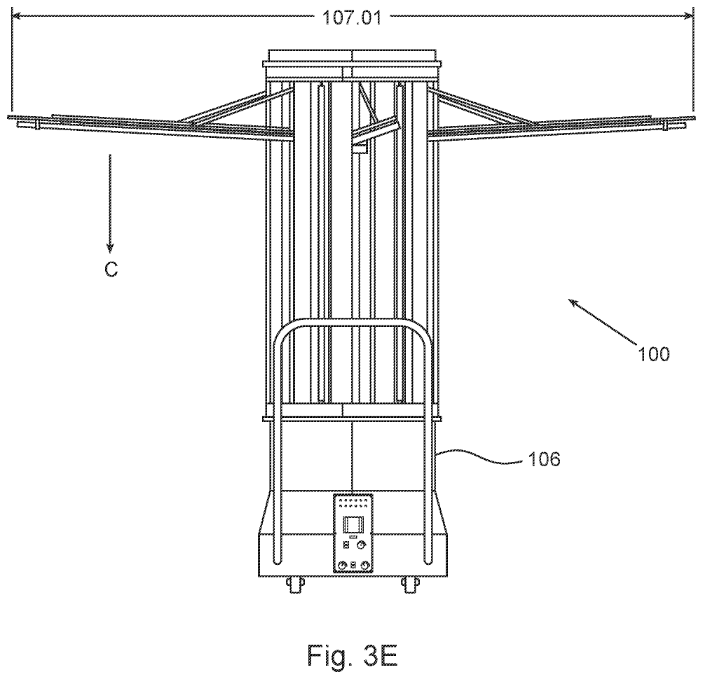

In an optional embodiment, the housing 106 includes shutters 302 that are movable between open and closed positions. In the optional embodiment illustrated in the figures, the shutters 302 may pivot or slide to expose or shield lamps 103. Several embodiments of operation of shutters 302 are described herein, and attention is directed towards FIGS. 3A, 3B, 20, 24A, 24B, 25A, and 25B.

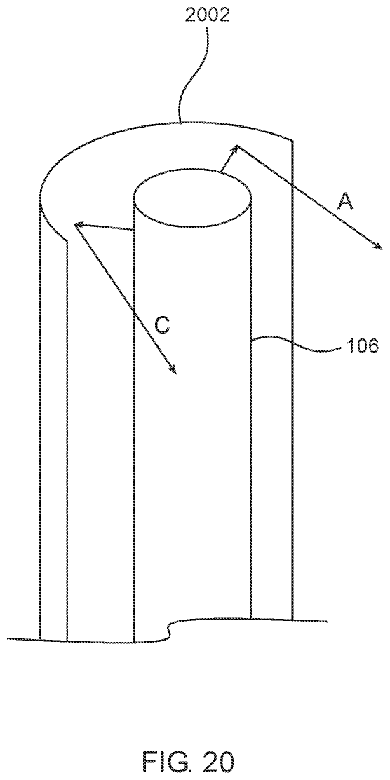

Attention is first directed to FIG. 20, which illustrates a close-up isometric view of static lamp 103 with parabolic reflector 2002 behind it to substantially direct all of the UV light generated by static lamp 103 outwardly from housing 106 of UVGI system 100 according to aspects of the embodiments (as denoted by Arrows A, B, and C). FIG. 20 illustrates one aspect of the embodiments of UVGI systems 100, 2200 wherein reflectors 2002 are located in such a manner in regard to lamp 103 such that substantially all of the UV light generated by lamp 103 is directed externally to UVGI systems 100, 2200 when shutters 302 are in the open condition. A first aspect of the embodiments of operation of shutters 302 are shown and described in regard to FIGS. 24A and 24B.

FIG. 24A illustrates a top partial view of UVGI Systems 100, 2200 with a movable shutter system in a closed condition according to aspects of the embodiments and FIG. 24B illustrates a top partial view of UVGI Systems 100, 2200 with a movable shutter system in an open condition according to aspects of the embodiments. In FIG. 24A, shutters 302a,b are in a closed condition; that is, they are in substantial alignment with the exterior of housing 106, and form a substantially smooth continuous surface thereof. If lamp 103 were to be inadvertently activated, its UV light would be substantially blocked by shutters 302a,b. Thus, shutters 302a,b protect lamps 103 when not in use, from dust and inadvertent damage, and shutters 302a,b protect people in the event of an inadvertent activation of lamps 103 when people are in the vicinity.

When instructions or commands are received from control panel 108, or via a remote interface, as described herein, to activate lamps 103, one of the instructions can be to open shutters 302a,b. In such case, commands are directed to shutter motors 2404a,b to rotate, in the direction of Arrows A shutters 302a,b about shutter hinges 2402a,b, respectively. Optional reflector 2002 can be included to reflect as much of the generated UV light outwardly through an arc of angle θ, as shown in FIG. 24B. According to further aspects of the embodiments, shutters 302a,b could be a single component, that is a single piece of appropriately shaped metal or other material that forms the substantially continuous outer surface of housing 106, and thus there might be only one hinge 2402, and one motor 2404, or there could be two or more motors for the single hinge 2402; such mechanical configurations are substantially unlimited in design and the above have been included to illustrate in a non-limiting manner the aspects of the embodiments.

Lamps 103 are static in the sense that they are relatively motionless in regard to housing 106. Shutters 302 can cover static lamps 103 when UVGI system 100 is operating in an alternate embodiment, described in greater detail herein.

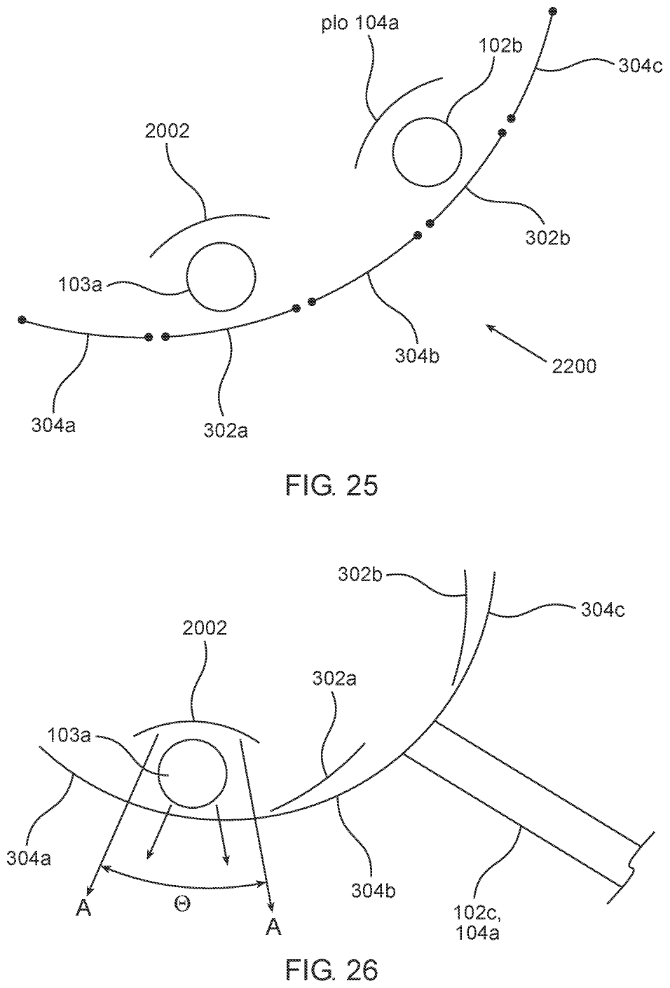

FIG. 25 illustrates a partial exploded view along lines A-A as shown in FIG. 3A according to aspects of the embodiments, and FIG. 26 illustrates a partial exploded view along lines B-B as shown in FIG. 3B according to aspects of the embodiments. As described herein, housing 106 can include both movable shutters (shutters) 302 and fixed slats (slats) 304.

FIGS. 25 and 26 illustrate the embodiments in which both dynamic and static lamps 102, 103 are used in either or both of UVGI systems 100, 2200. FIG. 25 illustrates the condition of shutters 302 as in FIG. 3A; UVGI system 100, 2200 is closed; this does not necessarily mean not operating, as either or both of lamps 102, 103 can be on. According to further aspects of the embodiments, the interior surface of housing 106, i.e., the interior surfaces of shutters 302 and slats 304 can be coated with a reflective surface such that any UV light transmitted from lamps 102, 103 are reflected inwards of housing 106 of UVGI system 100, 2200. In this case, the mechanical interface between shutters 302 and slats 304 is such that no or substantially no UV light is allowed to escape from the interior of UVGI system 100, 2200. In FIG. 25, lamp 103 has on one side shutter 302 and on the other side, substantially opposite to that of shutter 302 is reflector 2002. Adjacent to each side of shutter 302a are slats 304a,b. Adjacent to slat 304b is shutter 302b, behind which is lamp 102—the dynamic or moving lamp that is attached to arm 104. In FIG. 25, the arms 104 are in a stored, locked condition. Referring now to FIG. 26, slat 302a has moved out from its light blocking position in front of lamp 103a such that all or substantially all of the light generated by UV lamp 103 can be transmitted directly outward or reflected off reflector 2002 through the opening created by movement of shutter 302a in the direction of Arrows A in an arc of e. Similarly, shutter 302b has also moved out of the way of lamp 102a, so that arm 104 can extend and rotate upwardly with lamp 102a (in a manner previously described) and its UV light can transmit in substantially any direction depending on the orientation of lamp 102.

When in the open position, shutters 302 expose lamps 103 adapted to emit UV light. As discussed above, when in the closed position, shutters 302 substantially shield the lamps 103. Although the exact specifications of the lamps 103 may vary depending on the embodiment and the particular application, in one optional embodiment, the lamps 102, 103 are adapted to emit UV-C light having a wavelength of between 100-294 nm and an energy of between 4.43-12.4 electron volts (eV) per photon. As those of skill in the art can appreciate, ultraviolet (UV) light is a component of the electromagnetic spectrum that falls in the region between visible light and X-Rays. This invisible radiation includes the wavelength range of 100 nm to 400 nm. UV light can be further subdivided and categorized into four separate regions: 100 nm to 200 nm—Far UV or vacuum UV (these wavelengths only propagate in a vacuum); 200 nm to 280 nm—UVC (or UV-C)—useful for disinfection and sensing; 280 nm to 315 nm—UVB—useful for curing, and medical applications; and 315 nm to 400 nm—UVA (or “near UV”)—useful for printing, curing, lithography, sensing and medical applications.

As discussed above, DNA sustains damage at wavelengths of 185 nm and 265 nm. As such, conventional UV lamps 102,103 emitting UV light at wavelengths of 185 nm and 254 nm may be used in the present invention. This should be interpreted as illustrative rather than limiting since any lamp configured to emit UV-C light within a range of wavelengths between 100 nm and 294 nm may be used. It is contemplated that lamps 102,103 may be conventional fluorescent light bulbs, light emitting diodes, or a combination thereof.

In a first configuration, UVGI system 100 may be configured to disinfect and/or sterilize surfaces. In one such optional embodiment, at least one of the lamps 102 is mounted to an extendable arm 104. It is contemplated that all the lamps 102 may be mounted to extendable arms 104, or some lamps 102 may be mounted to extendable arms 104 while other lamps 102 are held stationary, such as by mounting them in a substantially fixed orientation to the housing 106. In yet other optional embodiments, lamps 102 may be mounted to extendable arms 104 in various orientations, such as an optional embodiment in which certain lamps 102 are oriented in an opposite direction as other lamps 102, such that some lamps 102 are oriented into the housing 106 while other lamps 102 are oriented out of the housing 106. In one such optional embodiment, some lamps 102 may be swept upward by extendable arms 104 while other lamps 102 are swept downward by extendable arms 104 as extendable arms 104 move/rotate.

The extendable arm 104 may redirect the lamp 102 to thereby redirect the UV light emitted from the lamp 102 as the extendable arm 104 moves. In an optional embodiment illustrated in the Figures, the lamp 102 is moveable between an orientation in which the UV light is emitted substantially horizontally from the housing 106, i.e., parallel to a ground level, and an orientation in which the UV light is emitted substantially vertically, i.e., perpendicular to a ground level. That is, at least some lamps 102 may be mounted to an extendable arm 104 that sweeps the lamp 102 ninety-degrees between a vertical lamp position, in which UV light is emitted horizontally from the lamp 102, and a horizontal lamp position, in which UV light is emitted vertically from the lamp 102.

In one optional embodiment, some lamps 102 may remain stationary with respect to the housing 106 while other lamps 102 move with respect to the housing 106. In an alternate optional embodiment, all lamps 102 may move with respect to the housing 106. In yet another optional embodiment, the lamps 102 may remain stationary with respect to the housing 106 and the housing 106 may move to thereby redirect emitted UV light. It is also contemplated that lamps 102 may be movable in different directions. For example, in one optional embodiment, certain lamps 102 may be movable from a vertical position to a horizontal position in which UV light is emitted upward, while other lamps 102 may be movable from a vertical position to a horizontal position in which UV light is emitted downward.

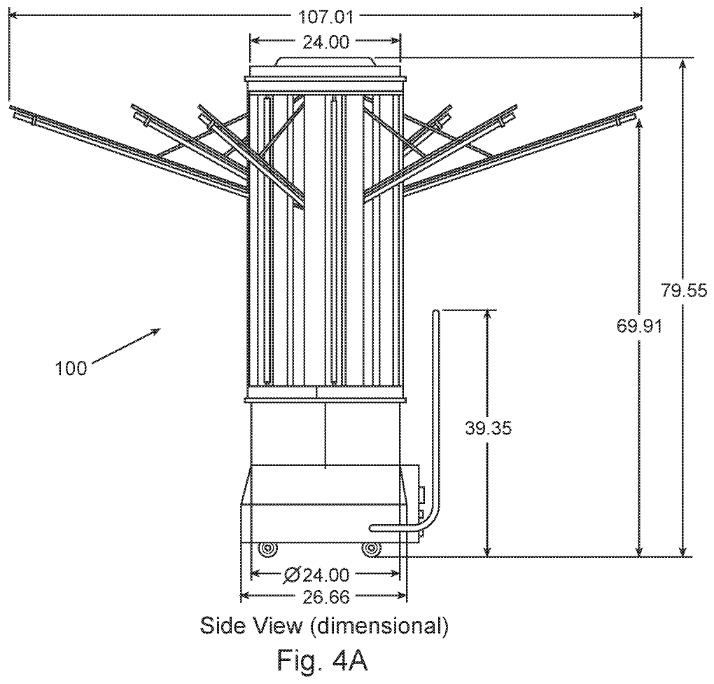

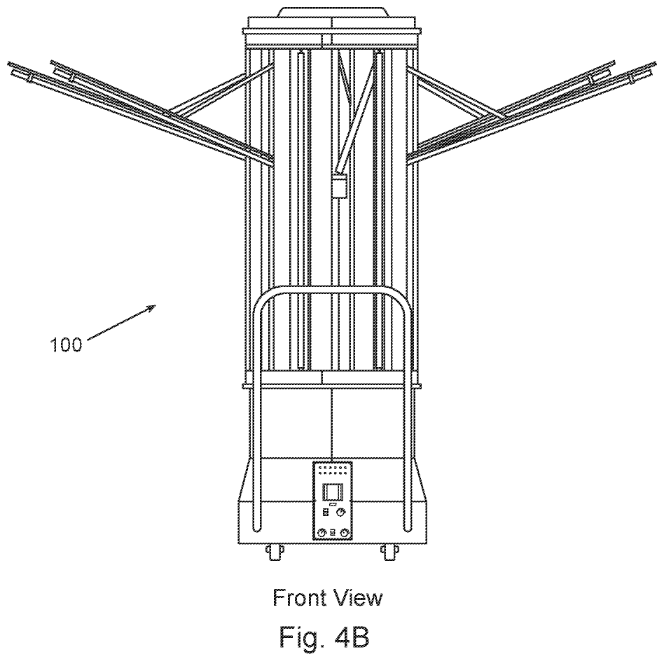

For example, in FIGS. 1 and 3-5 UVGI system 100 and extendable arms 104 are illustrated in various configurations as lamps 102 are exposed and carried by the extendable arms 104 through a range of orientations. The Figures also illustrate a configuration in which shutters are closed, thereby shielding lamps 102 from the environment. As discussed in greater detail below, the lamps 102 in such a configuration may be directed toward an air passage within the housing 106 to thereby treat air passing through the air passage.

FIGS. 3A through 3E are a series of schematic diagrams of UVGI system 100 shown in FIG. 1 according to an embodiment of the present invention, in which shutters 302 move from a closed condition to an opened condition, and wherein extendable arms 104 moved from a stored condition to an extended condition, carrying the lamps 102 out of the housing 106. In an optional embodiment, such a configuration may include at least some static lamps 103 directed outward from the housing 106 such that UV light is emanated substantially horizontal, e.g., parallel to a ground level.

FIGS. 3A through 3E illustrate a series of configurations of UVGI 100 in which the movable/extendable arms 104 carry the lamps 102 out of the housing 106, with the lamps 102 beginning to pivot from the housing 106 (FIG. 3C, in the direction of Arrow A). In this optional embodiment, the lamps 102 pivot downward from a vertical orientation to sweep UV light from the lamps 102 from horizontal, e.g., parallel to a ground level, to vertical, e.g., perpendicular to a ground level. That is, the lamps 102 can move from their stored, but open position of FIG. 3B, such the UV light transmits in the direction of Arrows A, and then, as shown in FIGS. 3C and 3D, extendable arms 104 rotate in the direction of Arrow B, such that the UV light is not beginning to be transmitted to less than horizontal position, and in FIG. 3E, the extendable arms 104 are substantially completely extended, such that the UV light is transmitted from lamps 102 in a substantially downward vertical direction (Arrow C), towards the ground/floor. As described above, however, some lamps 102 can point vertically up, such that the UV light is transmitted in a substantially vertical upwards direction (opposite that of Arrow C), or, according to further embodiments, some lamps can be rotated at various angles such that UV light is transmitted from lamps 102 in a substantially circular pattern of about 360° about the plane formed by extendable arms 104 when substantially completely extended.

FIGS. 3-5 illustrate a series of configurations of UVGI system 100 in which movable/extendable arms 104 continue to carry lamps 102 through a sweep from a stowed configuration inside the housing 106. In these optional embodiments, the configurations include lamps 102 oriented substantially vertically, e.g., perpendicular to a ground level. It is noted that although the optional embodiment shows lamps 102 oriented to emit UV light substantially downward, it is contemplated that lamps 102 may be additionally or alternatively oriented to emit UV light substantially upward. Moreover, while the figures show extendable arms 104 physically moving lamps 102 through a range of orientations to sweep UV light through a range of directions, it is contemplated that the physical movement of the lamps 102 may be replaced, in whole or in part, by optical means, such as reflectors, mirrors, lenses, or the like, which sweep UV light from the lamps 102 through a range of directions.

Furthermore, FIGS. 3-5 illustrate a series of configurations in which the lamps 102 of UVGI system 100 may be returned from the open configuration to a stowed configuration, by retracting the lamps 102 by pivoting the extendable arms 104 in the reverse direction. That is, in an optional embodiment, a cycle may include the lamps 102 moving from a stowed configuration illustrated to an open configuration, then back to the stowed configuration.

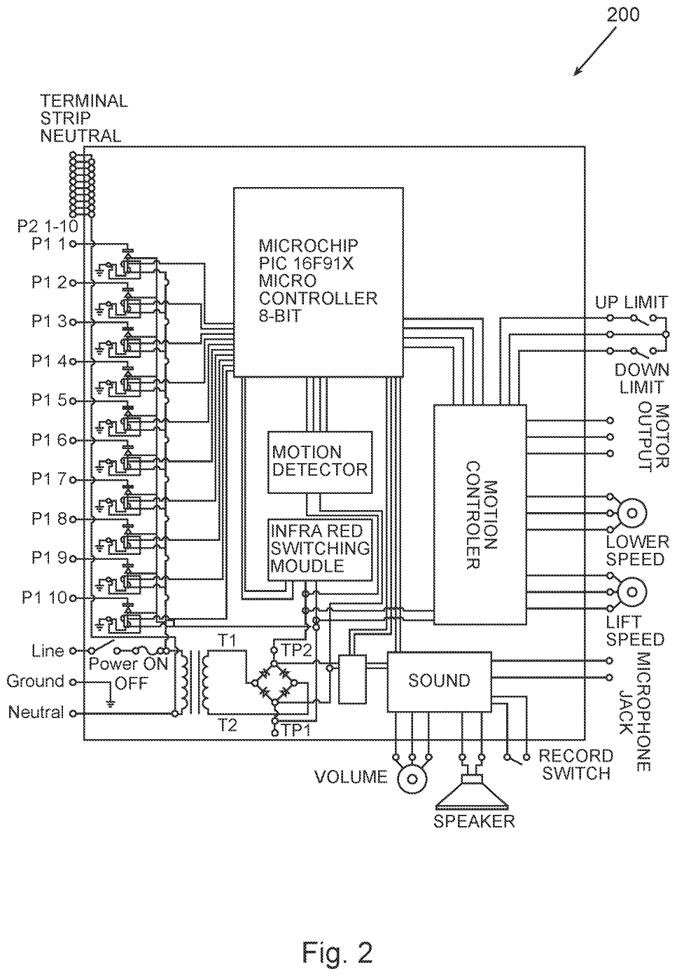

It is noted that the housing 106 and carriage 1702, if any, may cooperate to redirect UV light as well. For example, the housing 106 (or housing 106 and carriage 1702) may move linearly and/or rotationally to expose as much of the area to be disinfected/sanitized as possible. It is contemplated that the motion of the housing 106 or carriage 1702 and housing 106 may be controlled remotely, programmed into a controller 200 (shown in FIG. 2), or the like. Moreover, it is contemplated that the arms may move as the carriage 1702 travels to expose a defined space to UV light. In an optional embodiment, the carriage 1702 may include a motor controlled by a controller 200 to drive UVGI system 100 on a programmed path. In a further optional embodiment, the controller 200 may include memory that stores a programmed path and may communicate with sensors or other navigational aids such as radio frequency transceivers, global positioning receivers, proximity sensors, or the like, to provide motion and position guidance to the controller 200 and reduce the risk of collision with objects, walls, or the like. FIG. 2 is a schematic diagram of controller 200 for use in UVGI system 100 according to an embodiment of the present invention.

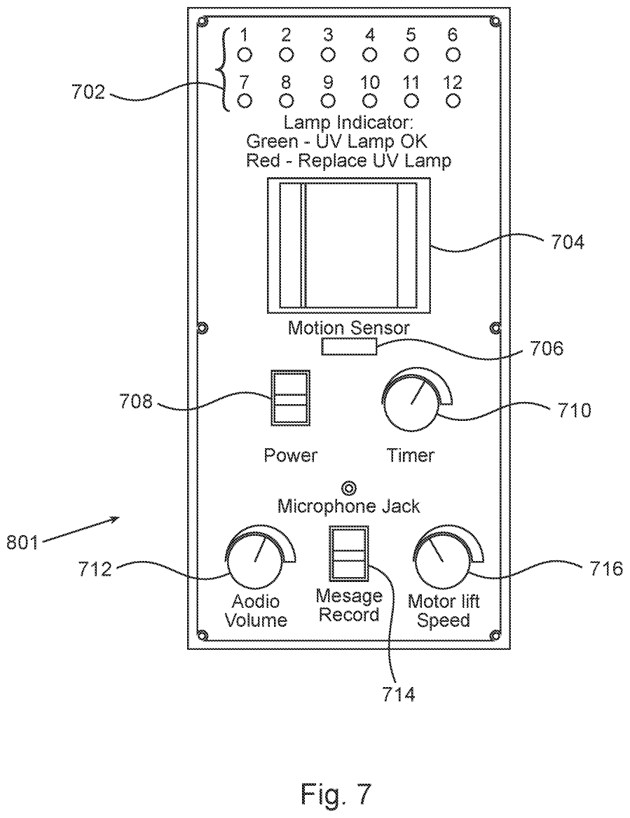

Controller 200 can communicate in a wired or wireless manner with control panel 108, as shown in FIG. 7. FIG. 7 illustrates control panel 108 for use with UVGI system 100 shown in FIG. 1 according to an embodiment of the present invention. Each of the items noted on control panel 108 communicates important information/data to controller 200 to operate UVGI system 100 according to aspects of the embodiments.

Control panel 108 comprises a plurality of lamp status indicators 702. There can be one lamp status indicator 702 for each of lamps 102, 103. A green color of lamp status indicator 702 can indicate an “OK” or “good” or “working” condition of the respective UV lamp, i.e., the respective lamp works and can transmit UV light, and a red color of lamp status indicator 702 can indicate a “bad” or “fail” or “not-working” condition of the respective UV lamp, i.e., the respective lamp does not work and cannot transmit UV light. Each of the lamps 102/103 can be numbered to correspond to respective lamp status indicators 702.

Control panel 108 further comprises a motion sensor display 704 and motion sensor switch 706. Motion sensor switch 706 enables control processor 200 to activate and monitor motion sensors (discussed below) for the presence of people in a nearby vicinity that could be harmed by the UV light transmitted from lamps 102,103. Display 704 shows when such motion occurs.

Control panel 108 further comprises power switch 708 and timer switch 710. Power switch 708 allows externally (115-240 VAC) or internally sourced (rechargeable battery) electrical power to flow to controller 200 and lamps 102,103, and other circuitry and motors, to be controlled by controller 200. Timer switch 710 can be a rotary potentiometer switch that provides a voltage that indicates an amount of time that UVGI system 100 should operate. According to further aspects of the embodiments, one, some or all of these functional inputs to controller 200 can be replaced by digital inputs, e.g., a keyboard, display, and the like, as known to those of skill in the art.

Control panel 108 further comprises audio volume switch 712, message record button 714, and microphone (mic) input jack 718. Together, devices 712, 714, 718 allow a user to attach an external mic to mic input jack 718 on control panel 108 of UVGI system 100, press button 714, record a message, such as a warning message, and have that message played back at the volume indicated by the setting of the volume switch 712 should a third party person or persons get too close to UVGI system 100 when it is transmitting UV light from one or more lamps 102,103.

Control panel 108 further comprises motor lift speed switch 716. Motor lift speed switch 716 sets the lift rate of extendable arms 104 with lamps 102 attached to them. [[Q: Please provide further information as to why this might be something that should be controllable]]

According to further aspects of the embodiments, any/all of the functions and capabilities provided by control panel 108 can be provided by a wirelessly connected control device (not shown) or a separate wired control device, and/or UVGI system 100 can be pre-programmed with such information that is retained by memory so that UVGI system 100 can operate substantially autonomously and retain data about its operations for analysis at later times. That is, UVGI system 100 can be controlled by any number of wired or wireless communications interfaces, including, but not limited to WiFi, Bluetooth, cellular communications, satellite communications, near field communication (NFC), and the like. UVGI system 100 can interface with the Internet or local networks as needed/desired through any/all of the wired/wireless communications interfaces (not shown).

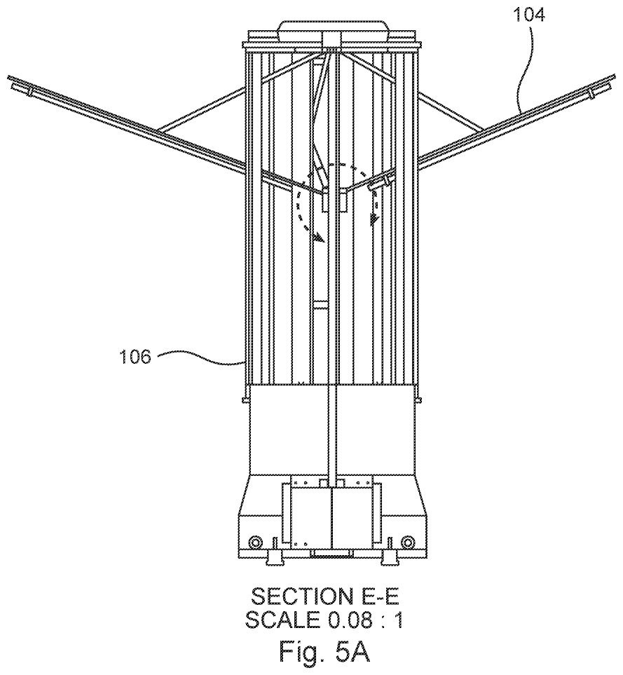

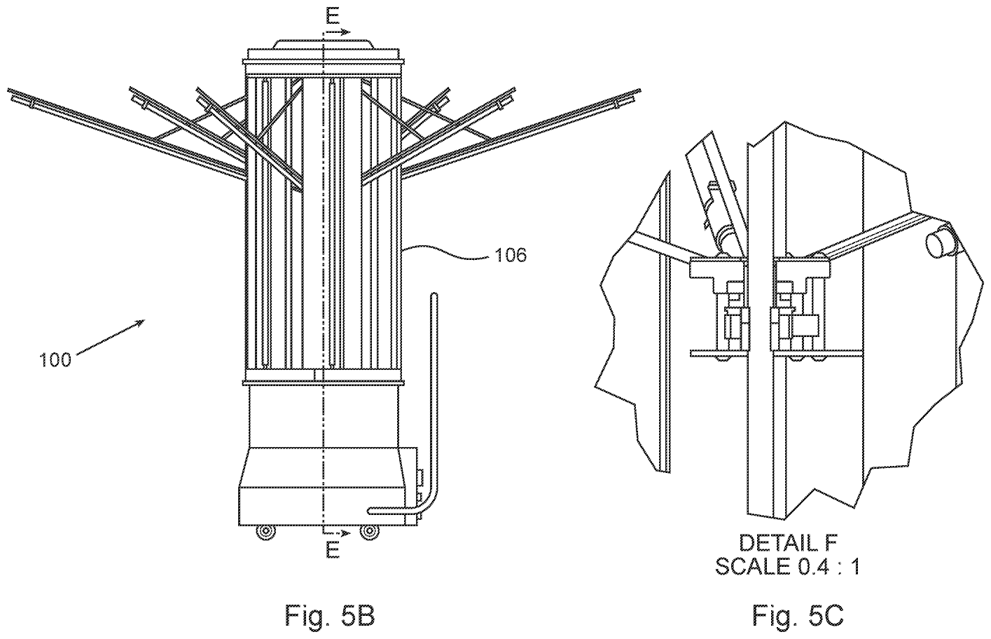

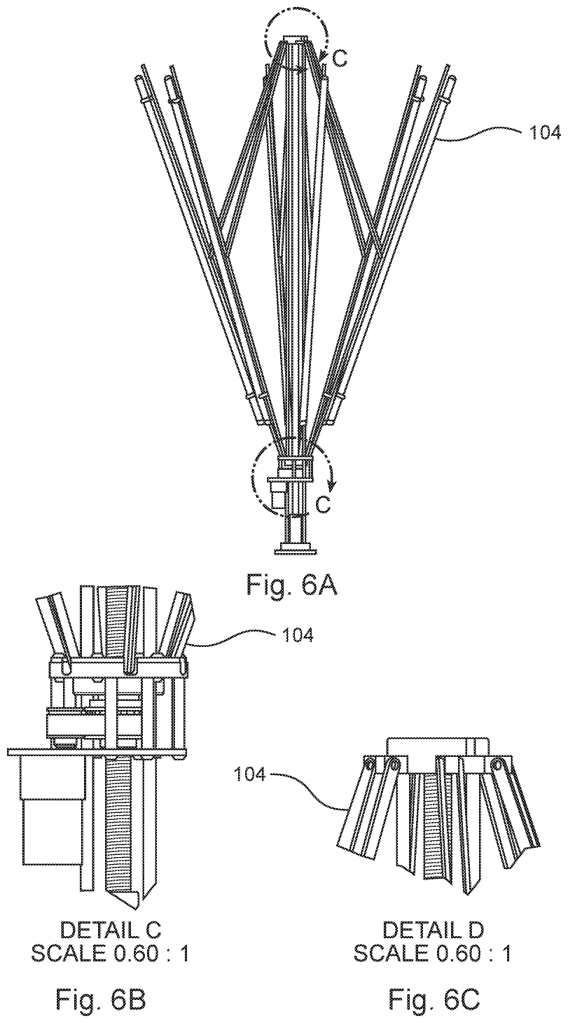

As illustrated in the Figures (see, e.g., FIGS. 5A-5C and 6A-6C) in an optional embodiment, extendable arms 104 may be mounted to the housing 106 through a pivot and motion that may be imparted to the extendable arms 104 through a drive connected to a drive link. In one such optional embodiment, the drive may include a trolley riding on a threaded shaft. The trolley includes internal threads so that the trolley is driven up and down the threaded shaft as the threaded shaft rotates. As the trolley moves up and down the threaded shaft, the extendable arms 104 mounted to the trolley pivot outward from the housing guided by a drive link connecting the housing 106 and extendable arms 104.

In an optional embodiment, the lamps 102, extendable arms 104, and shutters 302 communicate with a controller 200. In one optional embodiment, the controller 200 controls activation and deactivation of the lamps 102, including the timing of the activation and deactivation with respect to the positions of the shutters 302, orientation of the arms 102, and the like. Additionally, the controller 200 may control the duration of the activation of the lamps. In one such optional embodiment, the controller 200 includes a timer and a switch. In one such optional embodiment, the controller 200 is programmed to activate the lamps 102 during a time period measured by the timer and deactivate the lamps 102 after the time period measured by the timer expires. As may be appreciated, the time period may be input by a user (e.g., a user may set the time period for ten minutes) or may be calculated based on any of a number of variables (e.g., a time period of ten minutes may be stored in memory or calculated by a controller 200 for a room having a volume of forty cubic meters), among other methods for determining the time period the lamps are turned on.

In a further optional embodiment, the movement of the extendable arms 104 and the orientation of the lamps 102 mounted to the extendable arms 104 may be controlled by controller 200. In one such optional embodiment, a controller 200 may be programmed to control movement of the extendable arms 104 based on the time period measured by the timer. In one such optional embodiment, a controller 200 may be programmed to complete a defined set of movements. For example, the controller 200 may be programmed to move the extendable arms 104 at a speed so that a ninety-degree sweep of the lamps 102 from a vertical position to a horizontal position takes exactly the defined time period to complete.

In yet a further optional embodiment, a controller 200 may control the shutters 302. It is contemplated that a controller 200 may coordinate the shutters 302, extendable arms 104, and lamps 102 so that the shutters 302 are open, and lamps 102 are illuminated as the extendable arms 104 move through the programmed motions. In a further optional embodiment below, a controller 200 may coordinate the shutters 302 with an optional warning system and sensors.

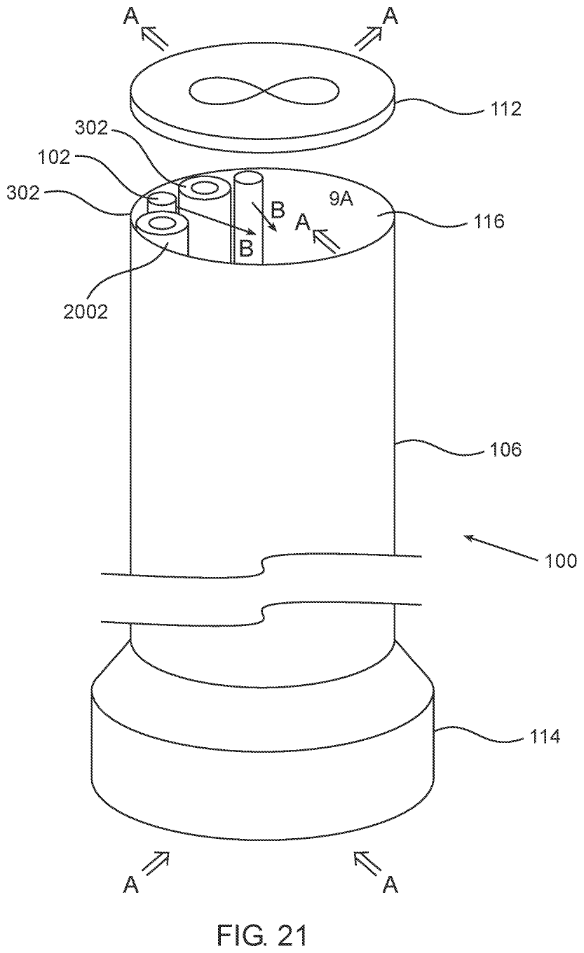

FIG. 21 is a partial cross sectional view of UVGI system 100 shown in FIG. 1 illustrating an alternate embodiment in which air is pulled into central air passage 166 of UVGI system 100 by one or more fans 112 and UV light from internally directed lamps 102 is used to disinfect/sterilize the air passing through central air passage 116 according to an embodiment of the present invention.

Controller 200 may also coordinate the lamps 102 and shutters 302 when UVGI system 100 is used in an alternate embodiment. That is, in an alternate embodiments, UVGI system 100 sterilizes/disinfects air by creating an air flow past lamps 102. In one such optional embodiment, closed shutters 302 cooperate with the housing 106 to direct UV light into an air passage, along with lamps 102 being set to their un-extended condition—meaning their storage position, within housing 106, such that the UV light from lamps 102 is directed inwards. In an optional embodiment, the air passage runs the length of the housing.

Referring now to FIG. 21, for example, in an optional embodiment, the housing includes a central air passage 116 that has an air inlet that communicates with ambient air (in this non-limiting example, air is drawn from the base 114 of UVGI system 100 in the direction of Arrows A). In the optional embodiment illustrated, the lamps 102 are directed toward the central air passage 116, with the shutters 302 substantially preventing light from escaping the housing 106. The lamps 102 may be aimed directly into the central air passage 116 or the light from the lamps may be redirected, e.g., reflected, into the central air passage 116, such that the UV light, shown as Arrows B, is directed inwardly, towards the center of central air passage 116. When the shutters 302 are closed, air is drawn through the central air passage 116 and exposed to UV light produced by the lamps 102.

In a further optional embodiment, the device may include a fan 112, compressor, or other mechanism for drawing air though the air passage past the lamps 102 and though an air outlet that communicates with ambient air. In an optional embodiment, the controller 200 controls the mechanism, e.g. fan 112, moving air through the central air passage 116. In the optional embodiment illustrated in the Figures (e.g., FIG. 21), a fan 112 is disposed at the top of a housing 106, with the air inlet near the base/carriage 114, 1702 of the UVGI system 100 and the air outlet near the top of UVGI system 100. Optionally, the air passage may include an air filter (not shown). As may be appreciated, the air filter may be disposed anywhere along the air passage. In the optional embodiment illustrated, an air filter may be disposed proximate the top of a housing near a fan 112. In one such optional embodiment, the air filter is positioned upstream from the fan 112, with the air filter exposed to UV light from the lamps 102. In this fashion, any microbe attached to particulate or liquid matter captured by the air filter may be exposed to UV light.

As discussed above, a controller 200 may be programmed to coordinate the various subsystems of UVGI system 100 to configure it for the air-cleaning mode. For example, controller 200 may be programmed to close the shutters 302, if they are not already closed, and actuate the fan 112 and the lamps 102. In this manner, ambient air may be drawn through the air inlet, exposed to UV light from the lamps 102 as the air passes through the central air passage 116, and ejected from UVGI system 100 by the fan 112 (note that in FIG. 21, the air is shown as being drawn up through central air passage 116; according to further aspects of the embodiments, the air can be moved in a downward direction if desired (i.e., in the direction opposite to that of Arrows A). In one such optional embodiment, this may occur upon receipt of a command; alternatively, this may occur as part of a pre-programmed sequence. For example, the controller 200 may be programmed to sterilize/disinfect the surfaces of a room in the first configuration discussed above, then sterilize/disinfect the air of a room in the second configuration discussed above.

Optionally, the controller 200 may include a remote activation system. In one such optional embodiment, the remote activation system could include a wireless transmitter and a wireless receiver. The wireless receiver is optionally in communication with the controller 200 and physically attached or part of UVGI system 100. The wireless transmitter can communicate via radio, ultrasonic, NFC transmissions, Bluetooth, or other wireless signal transmission methods with the wireless receiver. While the wireless transmitter may be configured for many commands and, optionally, two-way communication for status updates or the like, in an optional embodiment, the wireless transmitter is programmed to transmit at least an activation signal and a deactivation signal. As may be appreciated, receipt of an activation signal at the wireless receiver causes the controller 200 to execute a program to sterilize/disinfect a room and/or the air in the room and receipt of a deactivation signal causes the controller 200 to terminate the program.

In an optional embodiment, the controller 200 includes a shut off system to prevent UVGI system 100 from emitting UV light when a person is detected. In one such optional embodiment, the shut off system includes a sensor, such as a motion detector, and a switch connected to the lamps. Optionally, the controller 200 is programmed to open the switch, thereby deactivating the lamps, when the motion detector detects motion, or maintain the switch closed when no motion is detected.

Additionally, or alternatively, the controller 200 includes a warning system. In one such optional embodiment, the warning system includes visible and audible warnings for people who may be exposed to UV light. For example, in one optional embodiment, lights and/or lamps (which can be in the form of LEDs, conventional incandescent bulbs, and the like) that flash or otherwise warn of the activation or impending activation of the lamps 102 and speakers that broadcast warning sounds, recorded messages, or the like, may be provided. In one such optional embodiment, a storage device may store sound files that are broadcast by the controller 200 through the speakers.

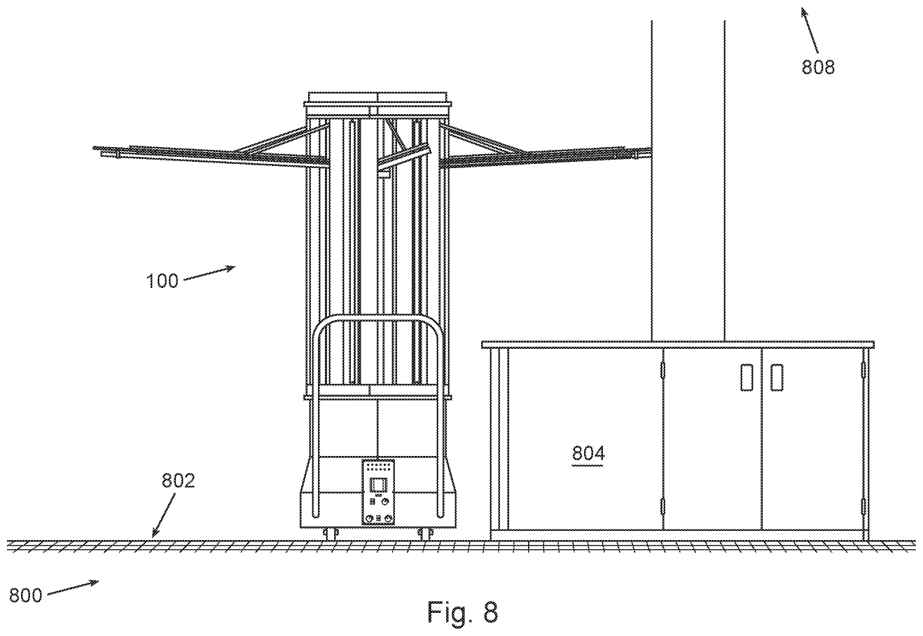

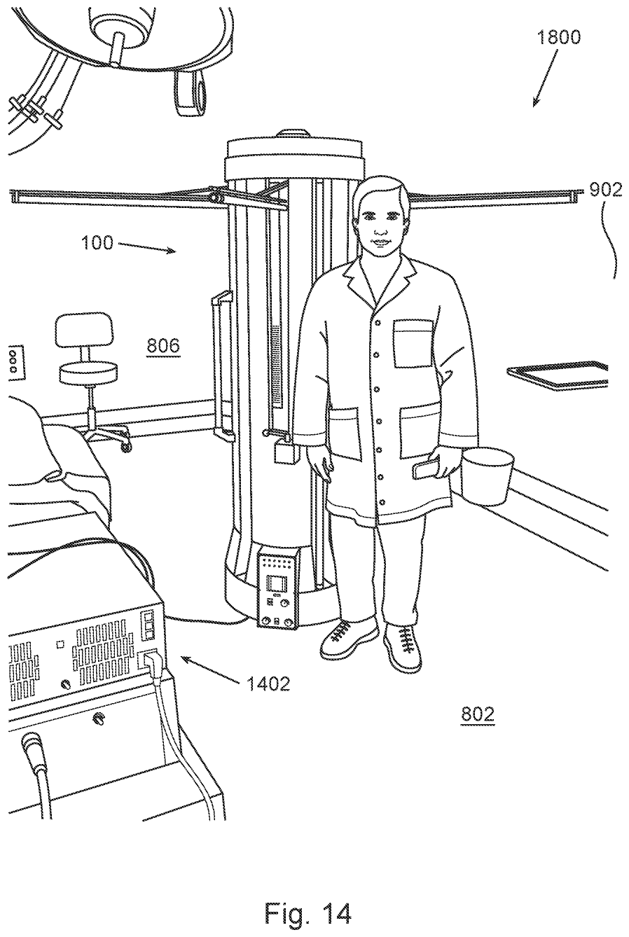

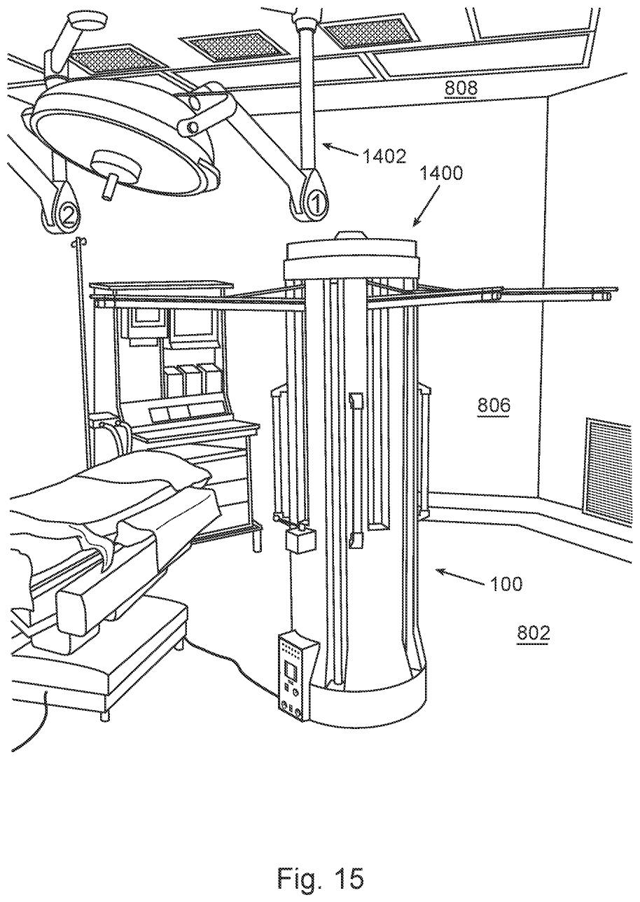

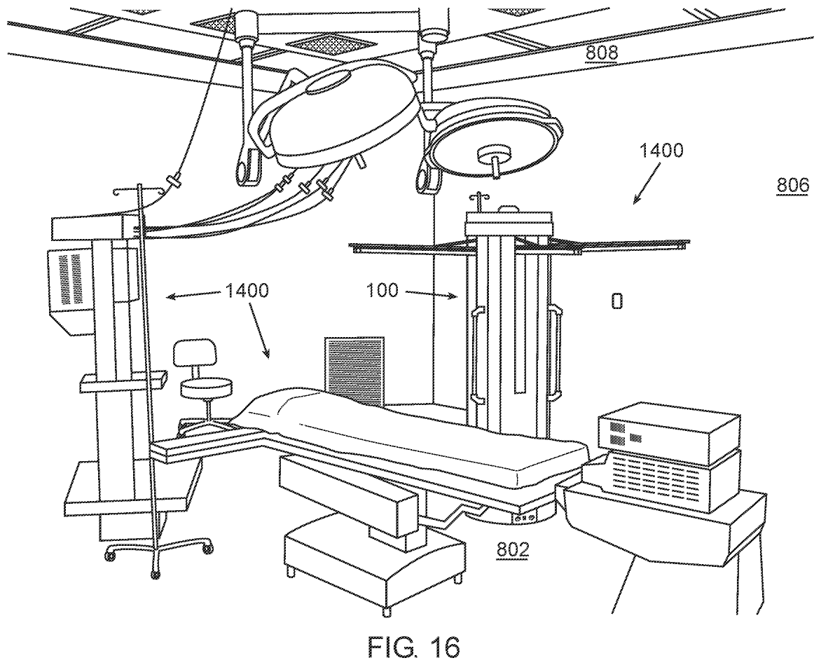

Attention is now directed to FIGS. 8-16. FIGS. 8-13 are schematic views of UVGI system 100 shown in FIG. 1 according to an embodiment of the present invention in a room such as an operating room; FIG. 14 is an illustration of UVGI system 100 shown in FIG. 1 according to an embodiment of the present invention in a “non-operating” or “off” condition while located in an operating room or surgical suite 1400 with surgical/medical equipment 1402; and FIGS. 15-16 are illustrations of UVGI system 100 shown in FIG. 1 according to an embodiment of the present invention in an “operating” or “on” conditions while located in an operating room.

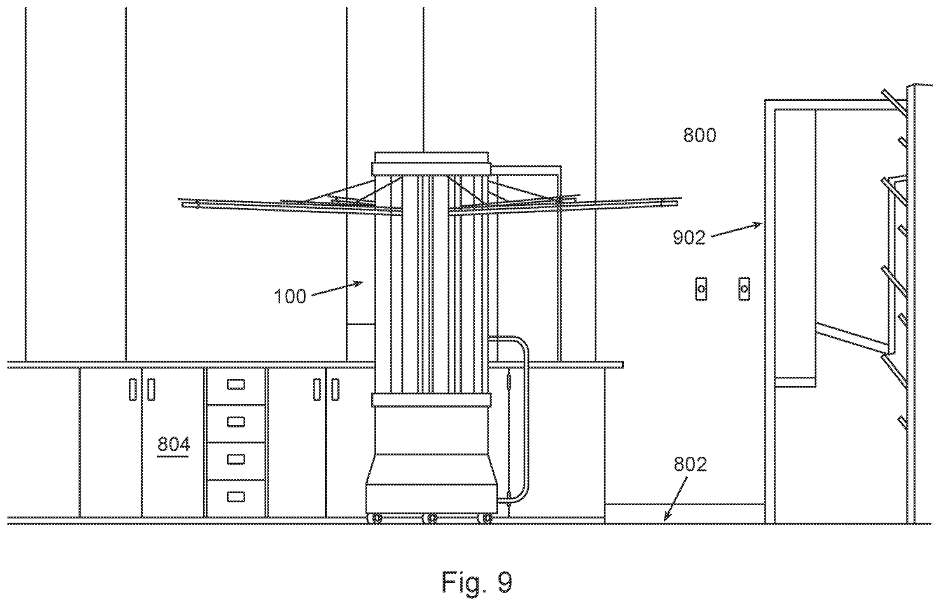

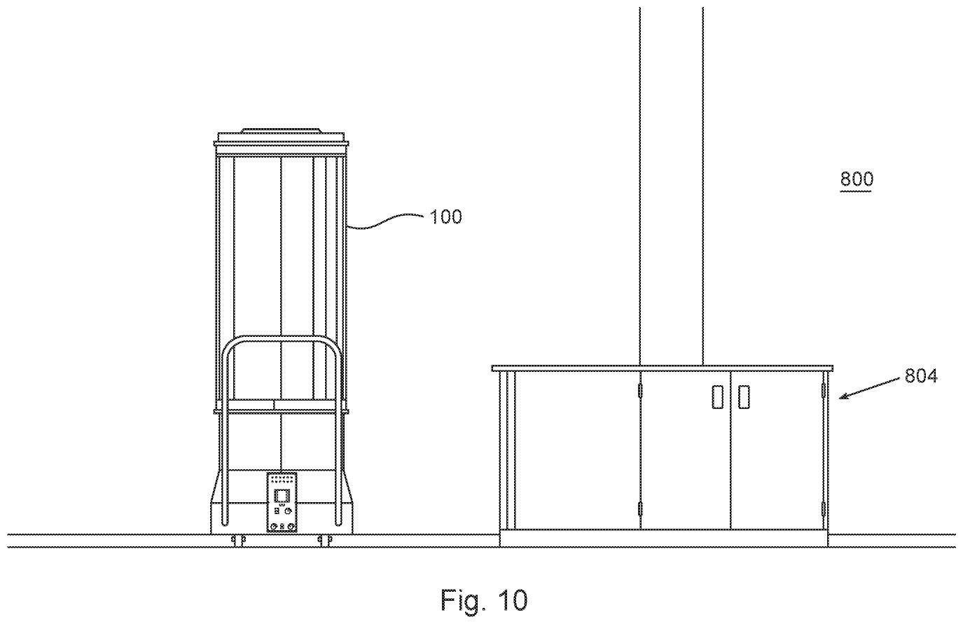

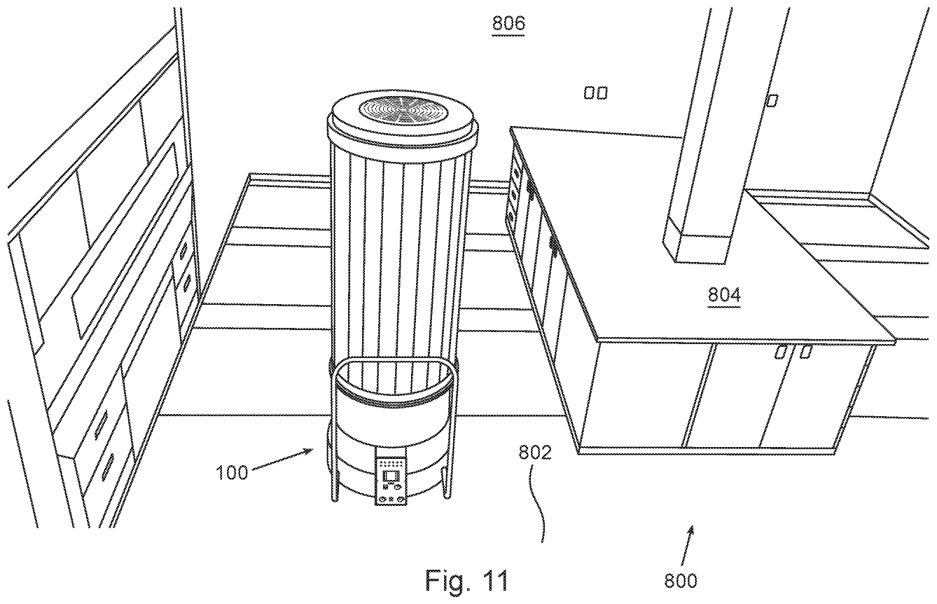

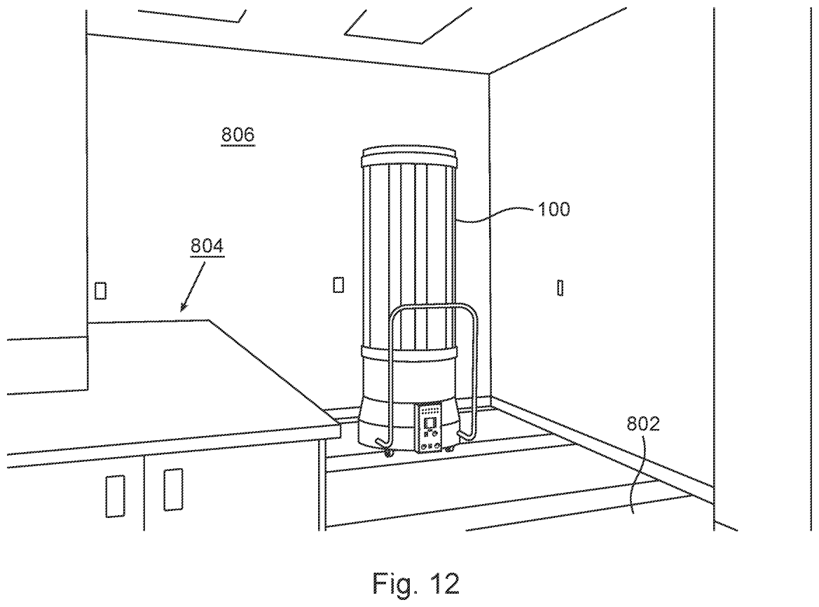

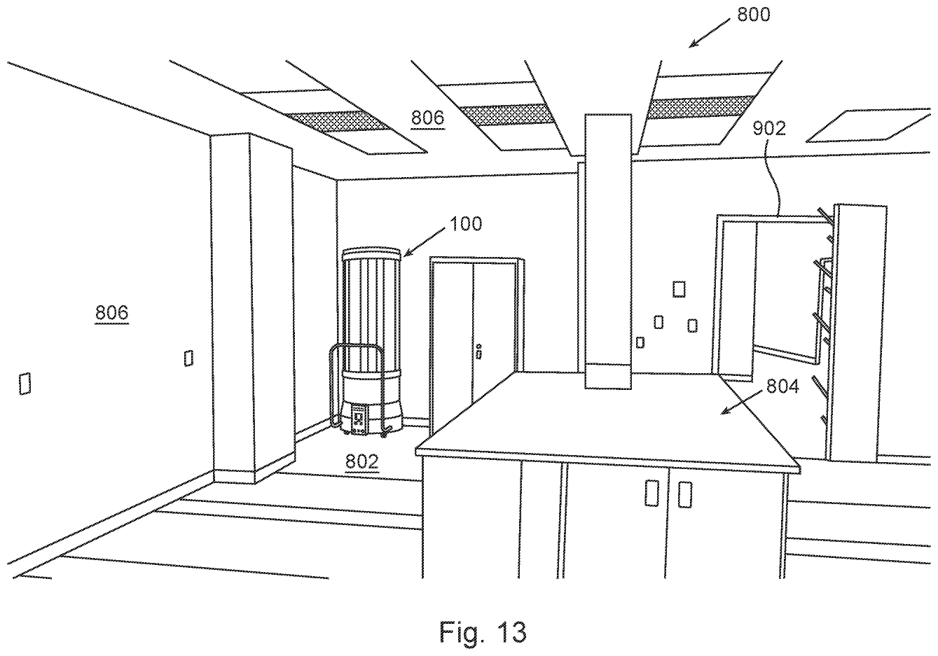

In FIG. 8, UVGI system 100 is located in a room 800, on a floor 802, with some furniture such as cabinets 804, walls 806 and ceiling 808. In the condition shown in FIG. 8, UVGI system 100 can be operating—i.e., transmitting UV light from the plurality of lamps 102 in the manner described herein. FIG. 9 is another view of room 800 and UVGI system 100 but includes door 902—in this condition, UVGI system 100 can be transmitting light from the plurality of lamps 102, but substantially constantly monitors the environment for the presence of humans that could enter through door 902. FIGS. 10-13 illustrates UVGI system 100 in a non-operating condition and as such can be placed in a corner for storage as shown in FIGS. 12 and 13.

In FIG. 14, UVGI system 100 is shown in another type of operating room, and is in a non-operating condition, as there is a human/physician located extremely close to UVGI system 100. However, in FIGS. 15 and 16, there are no people, and so all of dynamic lamps 102 and static lamps 103 are illuminated, transmitting UV light in an almost spherical pattern about UVGI system 100, substantially disinfecting/sterilizing the volume of air reached by the effective range of lamps 102/103 according to aspects of the embodiments.

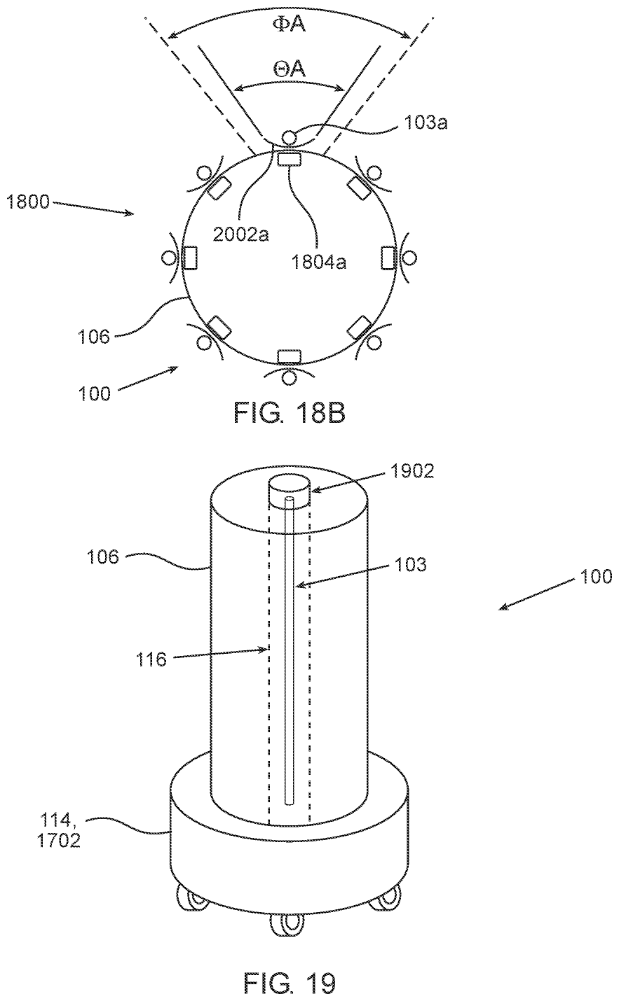

FIG. 17 illustrates a programmable remotely controlled self-propelled carriage (carriage) 1702) capable of moving UVGI system 100 shown in FIG. 1 according to an embodiment of the present invention on wheels; FIG. 18 shows a directional detection and avoidance system 1800 that can be used in the UVGI system 100 shown in FIG. 1 according to an embodiment of the present invention to prevent UVGI system 100 from exposing humans to UV light; and FIG. 19 shows the UVGI system 100 shown in FIG. 1 according to an embodiment of the present invention with central air passage 116 with UV light and high efficiency particulate air (HEPA) filter 1902 and fan 112 to clean the room air.

Shown in FIG. 17 is carriage 1702 capable of moving the UVGI system 100 on wheels. The movement of carriage 1702 is capable of autonomous or radio-controlled movement and can use wheels and other means to move UVGI system 100. The wheels can be replaced by tracks, or feet, or any combination thereof, as those of skill in the art can appreciate. Carriage 1702 receives its instructions from a controller located remotely (i.e., wired or wirelessly), or from controller 200 that is part of UVGI system 100.

FIG. 18A illustrates a partial front view of UV directional detection and avoidance system (system) 1800 that substantially prevents UVGI system 100 from exposing humans to UV light, and FIG. 18B illustrates a top view of UV directional detection and avoidance system 1800 according to aspects of the embodiments. Attention is directed to FIG. 18A. System 1800 comprises reflectors 2002a, static lamp 103, IR sensor 1804, all of which are attached to housing 106. UV light from static lamp 103 is directed outwards and also reflected off reflector 2002. The reflection of UV light from lamp 103 is also shown in FIG. 18B. FIG. 18B is a top view of system 1800. According to aspects of the embodiments, there can by any number of reflectors 2002 and respective static lamps 103; however, in this particular non-limiting embodiment, there are shown 8 such sub-assemblies of reflector 2002, static lamp 103 and IR sensor 1804. In this particular case, therefore, each reflector-lamp 2002-103 combination would cover—or transmit its UV light—over an area corresponding to an arc θ_{A} of about 45°. That is, reflector 2002a and lamp 130a spreads UV light through an arc θ_{A} of about 45°, and the same would be the case for the remainder of the sub-assemblies or reflector 2002, static lamp 103, and IR sensor 1804. Thus, both the lamp-reflector 103-2002 and IR sensor 1804 would encompass areas of coverage corresponding to about 45°.

As shown in FIG. 18B, IR sensor 1804a has a coverage arc of ϕ_{A}. ϕ_{A} and θ_{A} can be about the same. However, as those of skill in the art can appreciate, IR sensor 1804 can be designed to incorporate a slightly larger arc ϕ_{A} of coverage of about 50°, thus providing a larger “window” of protection to turn off its corresponding static lamp 103. These are but non-limiting examples included merely to facilitate discussion and understanding of the aspects of the embodiments. IR sensor 1804 shuts off UV light in that sector if a human being or other warm body is detected. The purpose is to avoid exposing people to UV light.

FIG. 19 shows central air passage 116 of housing 106 of UVGI system 100, wherein UV light from static lamp 103 and HEPA filter 1902 filter and disinfect/sterilize air from the area (room) that UVGI system 10 is located in according to aspects of the embodiments. According to aspects of the embodiments, UVGI system 100 can also include fan 112 at either a base 114/carriage 1702 region or at the top to draw air into central air passage 116 to clean the room air. According to aspects of the embodiments, use of HEPA filter 1902 and UV light from internally located static lamps 103 can substantially rid the air of bacteria, viruses, and dust.

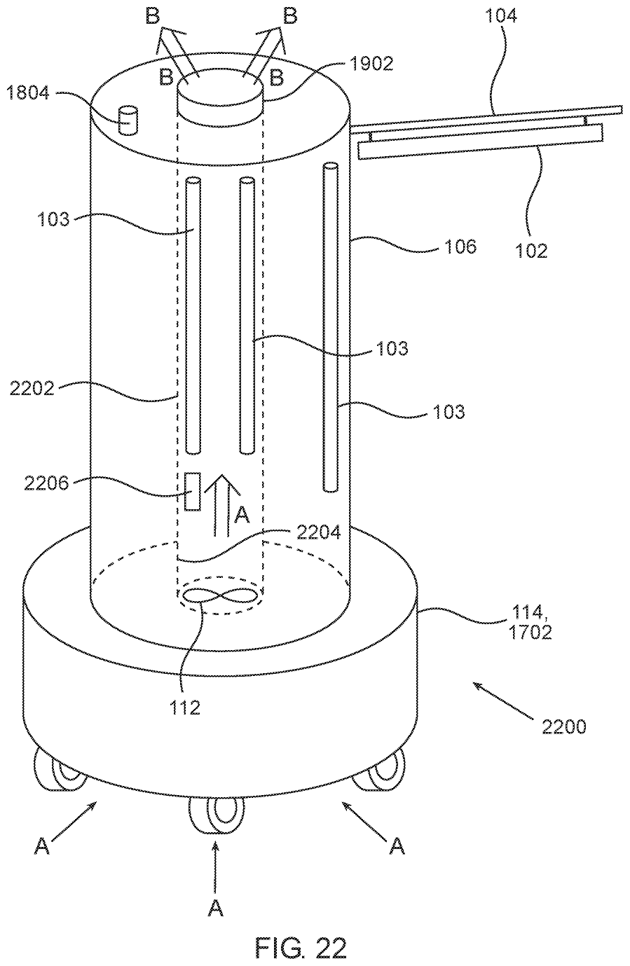

FIG. 22 illustrates a perspective view of UVGI system 2200 that includes additional features according to aspects of the embodiments. UVGI system 2200 is substantially similar to UVGI system 100 but includes additional features such as an internal central core air tunnel (tunnel) 2202, with internally co-located static lamps 103. Static lamps 103 illuminate the air with UV light as fan 112 pushes air through tunnel 2202, and which can then be filtered by HEPA filter 1902. In additional to the internally located static lamps 103, of which there can one or more, the internal surface of tunnel 2202, reflective inner surface 2204, is coated with a highly reflective material, or is made of a highly reflective material, such as stainless steel, which is not only reflective but easily cleaned by viral and other micro-organism killing solutions (e.g., bleach, and similar substances). Other materials that can be used as either reflective inner surface 2204 or to make up tunnel 2202 can include porcelain, nickel, aluminum, and gold plating. The properties of relative high reflectivity means that the one or more static lamps 103 can operate more efficiently in killing such microorganisms such as the Coronavirus, i.e., the disinfecting and/or sterilizing properties of the UV light is amplified with the implementation of highly reflective materials.

UVGI system 2200 further comprises heat sensor 2206. Because of the amplification of UV light transmission from the use of reflective inner surfaces 2204, heat can build up inside tunnel 2202, and excessive heat can create unsafe conditions. Heat sensor 2206 detects the temperature inside tunnel 2202 and generates either an analog or digital output signal. The output signal of heat sensor 2206 can be received and monitored by controller 200. If an over-temperature condition exists within tunnel 2202 controller 200 can temporarily shut down static lamps 103, and alert users of the condition.

UVGI system 2200 further comprises a safety switch that can be in the form of IR sensor 1804 that shuts down certain operations of UVGI system 2200 whenever the presence of a person is detected within a “danger zone” area about UVGI system 2200. According to further aspects of the embodiments, such a danger zone can include a radius of about 10-15′, or about 12-20′, or about 15-25′, or other ranges of radii within which a person exposed to UV light could suffer harmful side effects. The radii noted above are merely exemplary, and not to be taking in a limiting manner. Certain operations that would be shut down if one or more people entered the danger zone would be operation of the external lamps 102, 103. According to aspects of the embodiments, operation of the internally located static lamps 103 within tunnel 2202 could continue as substantially all, if not all or nearly all of the UV light transmitted by the lamps 103 located internally to tunnel 2202 is contained within tunnel 2202. According to further aspects of the embodiments, UVGI system 2200 further includes a plurality of either or both of lamps 102, 103 located on an external surfaces of UVGI system 2200.

In operation, as shown in FIG. 22, dirty, coronavirus laden air flows into UVGI system 2200 as shown by Arrows A, is cleaned/disinfected/sterilized within tunnel 2202 by the one or more internally located lamps 103, then expelled following filtering by HEPA filter 1902 as relatively clean air Arrow B.

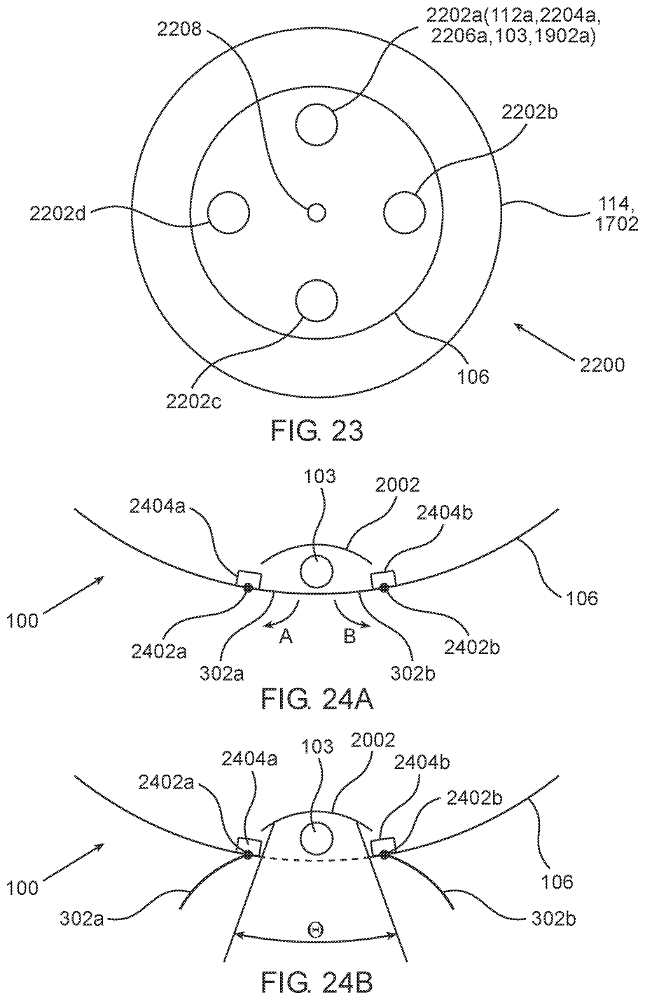

FIG. 23 illustrates a top view of UVGI system 2200 that includes further additional features according to aspects of the embodiments. Such additional features includes a plurality of tunnels 2202a-d. Each tunnel 2202a-d includes its own fan 112, reflective internal surface 2204, heat switch 2206, lamps 103, and HEPA filter 1902a. Each of these devices operates in a substantially similar manner as described above in regard to FIG. 22, among others, except in this embodiment there are two or more such tunnels 2202 and respective components. According to further aspects of the embodiments, there is shown one safety switch 2208, but this need not necessarily be the case. Additional switches 2208 can be used for redundancy and/or enhanced safety. While certain embodiments of the present invention have been shown and described it is to be understood that the present invention is subject to many modifications and changes without departing from the spirit and scope of the invention presented herein.

1. A device comprising:
a housing that comprises an air passage open to ambient air at both a top and bottom of the housing;
at least one internally located light emitting device adapted to emit ultraviolet (UV) light that is located completely within the air passage, and wherein UV light emitted from the at least one internally located light emitting device is substantially contained within the air passage;
at least one externally located light emitting device adapted to emit ultraviolet light externally from the device that is located on an exterior surface of the housing;
at least one infrared sensor adapted to determine a presence of a person within a first radius from the device and generate a warning signal if the person is located a distance away from the device that is less than or equal to the first radius; and
a controller adapted to control operation of both the at least one internally and externally located UV lights, and wherein when both are operating, the controller is adapted to shut off the at least one externally located UV light when the warning signal is received from the at least one infrared sensor.

2. The device according to claim 1, wherein the air passage contains highly reflective material.

3. The device according to claim 2, wherein the air passage is coated with the highly reflective material.

4. The device according to claim 2, wherein the air passage is made up of the highly reflective material.

5. The device according to claim 1, further comprising: a fan adapted to draw in externally located air into to the air passage, through the air passage, and then out of the air passage.

6. The device according to claim 5, further comprising: a filter adapted to filter the air prior to being expelled from the air passage.

7. The device according to claim 6, wherein the filter is a high efficient particulate air filter.

8. The device according to claim 1, wherein the UV light is further adapted to disinfect the air within the air passage.

9. The device according to claim 1, wherein the UV light is further adapted to sterilize the air within the air passage.

10. The device according to claim 1, further comprising: a base upon which the housing rests, the base adapted to make the device mobile.

11. The device according to claim 1, further comprising:
one or more extendable arms, each of the one or more extendable arms adapted to have at least one externally located UV light emitting device mounted on it, and
wherein the one or more extendable arms are adapted to be controlled by the controller to be moved from a storage position to a fully extended position, or any extended position in between the storage position and the fully extended position, so that UV light emitted from the externally located UV light emitting device can be projected outwardly from the device in a plurality of different directions based on the extended position.

12. A device comprising:
a housing that comprises an air passage open to ambient air at both a top and bottom of the housing;
at least one internally located light emitting device adapted to emit ultraviolet (UV) light located completely within the air passage, and wherein the UV light from the at least one light emitting device is substantially contained within the air passage, and further wherein the UV light is adapted to at least disinfect the air of viruses that is forced through the air passage; and
an inner wall of the air passage that contains a highly reflective material that is highly reflective of UV light, such that the air passage is further adapted to amplify the disinfecting properties of the UV light due to the reflective material that reflects the UV light within the air passage.

13. The device according to claim 12, wherein the air passage is coated with the highly reflective material.

14. The device according to claim 12, wherein the air passage is made up of the highly reflective material.

15. The device according to claim 12, further comprising: a fan adapted to draw in externally located air into to the air passage, through the air passage, and then out of the air passage.

16. The device according to claim 15, further comprising: a filter adapted to filter the air prior to being expelled from the air passage.

17. The device according to claim 16, wherein the filter is a high efficient particulate air filter.

18. The device according to claim 11, wherein the UV light is further adapted to sterilize the air within the air passage.

19. The device according to claim 11, further comprising: a base upon which the housing rests, the base adapted to make the device mobile.

20. The device according to claim 1, further comprising:
one or more extendable arms, each of the one or more extendable arms adapted to have at least one externally located UV light emitting device mounted on it, and
wherein the one or more extendable arms are adapted to be controlled by the controller to be moved from a storage position to a fully extended position, or any extended position in between the storage position and the fully extended position, so that UV light emitted from the externally located UV light emitting devices can be projected outwardly from the device in a plurality of different directions based on the extended position.

21. A device comprising:
a housing that comprises an air passage open to ambient air at both a top and bottom of the housing;
at least one internally located light emitting device adapted to emit ultraviolet (UV) light that is located completely within the air passage, and wherein UV light emitted from the at least one internally located light emitting device is substantially contained within the air passage;
at least one heat sensor adapted to sense a temperature inside the air passage and generate and transmit a temperature signal; and
a controller adapted to control operation of the at least one internally located UV light emitting device, and wherein when the at least one internally located UV light emitting device is operating, the controller is adapted to shut off the at least one internally located UV light emitting device when the temperature signal received from the at least one heat sensor indicates an over-temperature condition.
